(19) United States
(12) Patent Application Publication
(10) Pub. No.: US 2016/0271208 A1
(43) Pub. Date: Sep. 22, 2016

(54) ROOT CAUSE CURE AND PREVENTATIVE MEASURE FOR SCHIZOPHRENIA AND OTHER MENTAL ILLNESS

(71) Applicant: Richard D. TUCKER, Locust, NC (US)

(72) Inventor: Richard D. TUCKER, Locust, NC (US)

(21) Appl. No.: 15/073,719

(22) Filed: Mar. 18, 2016

Related U.S. Application Data
(60) Provisional application No. 62/135,969, filed on Mar. 20, 2015.

(51) Int. Cl. A61K 38/16; A61K 45/06

(52) U.S. Cl. CPC A61K 38/16; A61K 45/06

(57) ABSTRACT

A method and system for treating schizophrenia and other forms of mental illness, including: given a brain comprising neurons coupled by an axon including an inner core and an outer myelin sheath, and given one or more defects in the outer myelin sheath, repairing the one or more defects in the outer myelin sheath with one or more of a protein and a lipid such that the outer myelin sheath has a substantially constant electrical impedance for the transmission of data energy between the neurons and such that data energy is not undesirably reflected from the direction of a receiving neuron in the direction of a transmitting neuron within the axon.

### CROSS-REFERENCE TO RELATED APPLICATION

The present patent application/patent claims the benefit of priority of co-pending U.S. Provisional Patent Application No. 62/135,969, filed on Mar. 20, 2015, and entitled “ROOT CAUSE CURE AND PREVENTATIVE MEASURE FOR SCHIZOPHRENIA AND OTHER MENTAL ILLNESS,” the contents of which are incorporated in full by reference herein.

### FIELD OF THE INVENTION

The present invention relates generally to a method for curing the root cause of schizophrenia and other forms of mental illness. More specifically, the present invention relates to neurological procedures for supplanting defective genes and associated proteins with correct genes and associated proteins such that the proper magnitude of activation of the genes and associated protein expressions correct defective communications between neurons.

### BACKGROUND OF THE INVENTION

Techniques for reducing the effects of schizophrenia and other forms of mental illness are known. However, obtaining a complete cure of schizophrenia and other forms of mental illness has not been accomplished. Prior methods have attempted to reduce the effects of schizophrenia and other forms of mental illness by modifying the transmission and reception chemistry of the brain neurons' data energy to reduce the ill-timed and confusing data messages between neurons; however, this prior art requires medication causing the side effects of tremors, repetitive ticks, and general isolation of the patient from reality and does not address the root cause of schizophrenia. To date, doctors and medical researchers do not have the answers to “what causes schizophrenia” or “how do you cure schizophrenia.” As such, there is a need in the art for medical systems and methods that repair the root cause of schizophrenia and other forms of mental illness.

Furthermore, prior art medical research has amassed significant data concerning the transmission and reception of data messaging between neurons, such as the chemistry of electron data packet passage, the speeds of the electron data packet passage with and without proper axon myelination, and the genes, proteins, and molecular structures responsible for proper myelination of the axons. There exists a need in the art to find a means to properly myelinate axons for optimum data packet transfer between designated communicating neurons.

Furthermore, prior art medical research has determined that neurons communicate via a mesh networking topology through the neuron axon multiple endings to dendrites of other multiple neurons and have needs of sophisticated timing to accurately transfer properly addressed data packets to the proper receiving ends and without any distortion. However, prior art medical research has not determined how improperly addressed messaging of mentally ill patients' brains' neurons lose track of where some data packets “should go” and where some data packets “have come from,” resulting in hallucinations in the form of “voices, visual scenes, pressure sensory feelings, and other imaginary but seemingly real mental representations.” The resulting furious overflow of extraneous improperly addressed data packets' energy along with the properly addressed normal data packets' energy causes neurotransmitter regulation to “turn back” the availability of neurotransmitters, which in turn induces the negative symptoms of schizophrenia and other mental illnesses. There exists a need in the art to find a means to remedy the improper generation of reflected and address void data packets between the mentally ill brains' neurons' to avoid the positive and negative symptoms of schizophrenia and other mental illnesses.

### BRIEF SUMMARY OF THE INVENTION

It is an object of the present invention to overcome deficiencies in the prior art by providing processes and components for the cure of schizophrenia and other forms of mental illness.

In various exemplary embodiments, the present invention provides a method and process for repairing the root cause of schizophrenia and other forms of mental illness. A healthy brain “neuron to neuron” communication consists of chemically induced electron travel along a connection between the neurons. This connection is an axon which has a core length specialized in cellular structure for passing the neuron data energy and which has a covering around this data passing core for insuring the efficient passage of the data energy.

This axon covering is formed by a process in the brain called “myelination,” resulting in a myelin sheath made of a modified plasma membrane consisting of lipids and proteins. The myelin sheath provides an electro-magnetically consistent impedance for the data energy to travel from Neuron A to Neuron B. With consistent electro-magnetic impedance along the path of the data energy, the speed of the electron travel can be optimized and theoretically could approach or equal the speed of light. Further, the longer the distance between the neurons, the more critical the need for the consistent impedance along the path provided by the myelin sheath. Indeed, as in electro-magnetic transmission of TV and radio stations, the coaxial cable is an extremely important function of providing the optimum and efficient path for the radio station transmission energy to the antenna, which is also optimized to match the impedance of the atmosphere and surrounding physical conditions. When a radio station coaxial cable (as with the axon myelin sheath) has a damaged spot along the path of the transmission data energy, a portion of the transmission energy is reflected to the transmission energy source (as in Neuron A). The radio station transmission efficiency to the antenna via the coaxial cable (as the axon covered by the myelin sheath) is determined by the “Standing Wave Ratio or SWR,” which is a ratio of the data energy sent to the antenna through the coaxial cable and the data energy reflected back to the data transmission source. As with the radio station data energy, the brain neuron transmission to another neuron has the same reflected data energy if there is a defect in the axon myelin sheath. The result of the reflected data energy between the brain neurons causes confusion as this reflected data energy arrives before Neuron A is expecting a reply from Neuron B. To explain this expected time of response from Neuron B, universally, the source data transmitter (Neuron A) will send a message to the sink receiver (Neuron B) and will expect a reply of acknowledgement of “receipt and understanding of message,” “receipt and no understanding of message,” or nothing after a specified length of time. The transmitter does not expect a reply before this length of time as it knows the timing of the exchange. The reflected data energy caused by the defect in the coaxial cable (axon myelin sheath) will arrive before the expected time of reply. The source transmitter (Neuron A) will not know what this data packet is as it is being received in a “no man's land” period of time.

As each neuron is connected in a mesh arrangement with other neurons' dentrites via axon multiple endings (not just between Neuron A and Neuron B), the mysterious and “unaccounted for” data packet is passed on to other “mesh connected” neurons in the effort to find the “proper owner” of this mysterious data packet. It is noted here that “mesh networks” operate in this fashion (passing on messages) that do not belong to that particular node (or neuron in this case) until the proper owner (node that the data packet final destination is addressed for) is found, then an “acknowledge message” is sent back through the network to the proper sending node (neuron) to complete the data packet “send/receive” transaction. It can be seen here that “resending of a data packet of unknown origin and of no proper destination addressing” within a mesh network can quickly create a cacophony. It is of no wonder that those affected by schizophrenia sometimes seek minimal sensory input (darkness and quiet) to keep the data packet cacophony at a minimum. The brain is a marvelous organ to manipulate and repurpose the reflected extraneous data within it into other data forms such as voices and or hallucinations for the afflicted person with the damaged or incompletely myelinated axons. In an exemplary embodiment, a method to complete the myelination of the axons of the afflicted person's brain will cure the schizophrenia root cause by eliminating the extraneous reflected data energy back to the source transmitter (Neuron A) and so eliminate the source of the voices and hallucinations.

Schizophrenia has positive and negative symptoms. Both, the negative and the positive symptoms are results of “over-dutied” mesh network communications, which in turn reduce the neurotransmitters' availability by “neurotransmitter regulator proteins” to slow the messaging synapse pathways. Negative symptoms in schizophrenia refer to a decrease or absence of normal function. An example of this is a loss of interest in everyday activities. Negative symptoms may be present years before positive symptoms in schizophrenia occur. Schizophrenia negative symptoms can be hard to diagnose as they can easily be mistaken for other disorders like depression. Negative symptoms in schizophrenia include:
1. Apparent lack of emotion or small emotional range
2. Reduced ability to plan and follow-through with activities
3. Neglect of personal hygiene
4. Social withdrawal, decrease in talkativeness
5. Loss of motivation

People with schizophrenia who have negative symptoms often need help with everyday tasks and with taking care of themselves. It can appear like the person with schizophrenia isn't trying or doesn't want help, but this is just a manifestation of his or her negative symptoms. Positive symptoms in schizophrenia refer to an excess or distortion or normal function. Positive symptoms are the ones most typically associated with schizophrenia or psychosis. These include:
1. Hallucinations: which are often auditory (often hearing voices). These symptoms are the ones that generally cause people to lose touch with reality. Positive symptoms of schizophrenia can come and go and may not be noticeable at times.
2. Delusions: falsely held beliefs usually due to a distorted perception or experience. Delusions are the most common symptom of schizophrenia.
3. Thought disorder: difficulty organizing and expressing thoughts. This might result in stopping mid-sentence or speaking nonsensically; including the making up of words.
4. Disorganized behavior: unusual and inappropriate behavior. This might be childlike behavior or unpredictable agitation.
5. Movement disorder: agitated or repeated movements . Catatonia (non-moving and non-responsive) is also possible.

Positive symptoms often respond more successfully to antipsychotic treatment of the prior art, however, eliminating the reflected and unaddressed data packets will eliminate the over-dutied brain neuron mesh network communications and will remove the need of the neurotransmitters' reduction and eliminate the symptoms of schizophrenia.

It is known that brain myelination occurs during several stages of human development from being an infant, young child, pre-teenager, and “late teenage to young adult years.” It is also known that the last and most important myelination is during the “late teenage to young adult years” where the human gene set produces a protein set that has the responsibility for two things:
1. Pare down the memory dendrites
2. Complete the myelination of the axon

It is also known that the paring of the memory dendrites and the completion of the myelination of the axons will not happen if this gene set is not capable by damage or other defect to produce the proper proteins. It is also known that schizophrenia occurs during that same period of time for teenagers and young adults that do not experience the final and proper myelination of their brain axons. This novel process and method to prevent and or cure schizophrenia and other mental illnesses may be accomplished by detecting the abnormal or damaged (it has been shown that certain virus or disease during pregnancy may damage the embryo genome) gene(s) of the schizophrenia patient or even scan young people for the abnormal or damaged myelination gene(s) and utilize gene splicing or other gene correcting methods to put into place and activate the proper gene(s) to produce the proper proteins for the memory dendrite paring and final and proper axon myelination.

Other objects and advantages of the present invention will become apparent to those of ordinary skill in the art upon review of the detailed description of the preferred embodiments and the attached drawing figures, in which like reference numerals are used to represent like components.

### BRIEF DESCRIPTION OF THE DRAWINGS

The present invention is illustrated and described herein with reference to the various drawings, in which like reference numbers are used to denote like system components/method steps, as appropriate, and in which:

FIG. 1 is a schematic diagram showing a communication system between a pair of brain neurons' signaling through a damaged axon myelination sheath indicating the root cause of schizophrenia and other mental illness and how that root cause can be corrected by the system and method according to an embodiment of the present invention; and

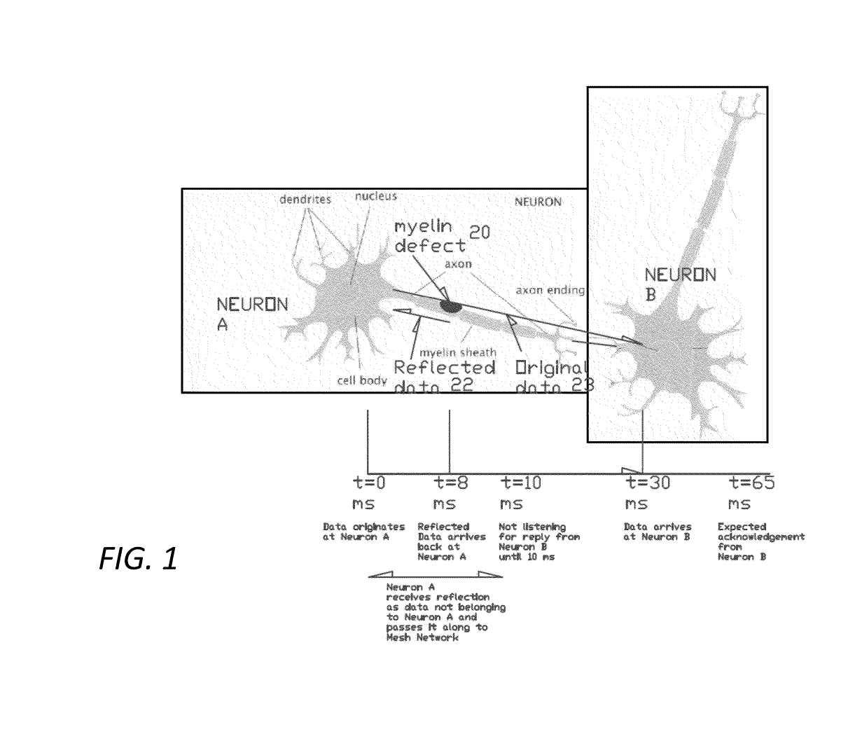

FIG. 2 is a schematic diagram showing a simple mesh network system built with multiple neurons, their axon multiple ends and the synapses connecting to multiple other neurons.

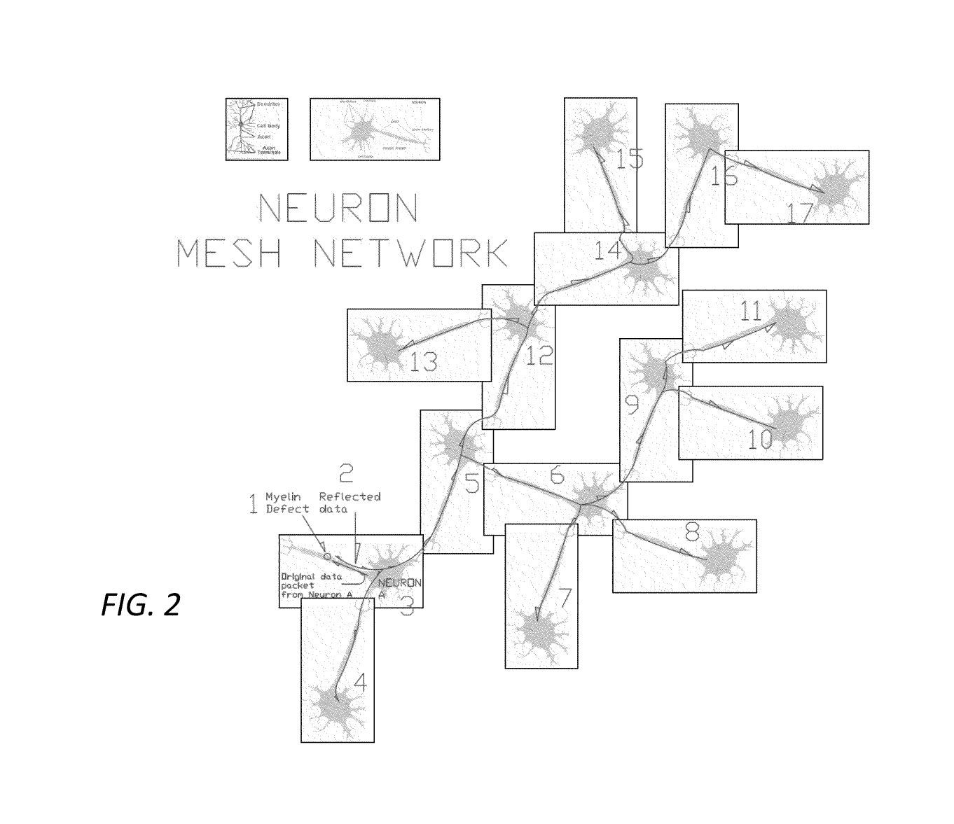

### DETAILED DESCRIPTION OF THE INVENTION

It is known in the medical research community that myelination of axons enables data packet “impulses” to travel from one cerebral hemisphere to the other typically in 30 milliseconds. This is compared to 150 to 300 milliseconds through “unmyelinated” axons. In some of the axons, the chemical action potential travels at a rate of 1.2 to 250 miles per hour. The myelination substance is manufactured in sheets by glial cells. An octopus-shaped glial cell called an oligodendrocyte does the wrapping somewhat like electrical tape up to 150 times between every segmented node of the axon. The segmented points between nodes of the myelination allow data repeating for maintaining the signal strength. Biologist Klaus-Armin Nave of the Max Planck Institute for Experimental Medicine in Gottingen, Germany, discovered that Schwann cells detect a protein called neuregulin that determines whether the Schwann cell wraps more or fewer sheets of myelin around the axon for the optimum thickness of the myelin insulation. It was also found that many people who suffer bipolar dis-order or schizophrenia have a defect in the gene that regulates production of the protein neuregulin. The medical research community have amassed all of the pertinent data for the neuron/axon myelination optimization, however, the root cause for disruptive data packet impulses of schizophrenia have eluded them, not because of inadequate brilliance, but merely because their training did not include electrical engineering communication transmission theory.

The characteristics of the myelinated axons and the characteristics of coaxial cables are undeniably the same. The medical research community's peer reviewed papers refer to the data packet energy passed from one neuron to another as “impulses.” We will also refer to the data packet energy as “data packet impulses.”

It is also known in the medical research community that without myelin, the data packet impulse (in their terms) leaks and dissipates. They have found that maximum conduction velocity requires strict proportional myelination insulation to the diameter of the bare axon fiber. The ratio of bare axon diameter divided by the total fiber diameter (including the myelin) is 0.6 for optimum data packet impulse speed along the axons.

The electrical data packet impulse travel through axons are bound by physical laws, physics and chemistry, thus, follow the same limitations of electronic coaxial cables that have defects concerning the change of impedance along the transmission route of the data packet energy. As it is seen in the following electrical engineering description of coaxial cable data packet impulse “reflection,” the same physics and chemistry can be applied to damaged, imperfectly applied, or genetically missed myelination of axons. Where the referenced frequency of the coaxial cable is noted, the frequencies of the data packet impulse for axon transmission would be calculated with the Fourier series analysis method based upon the mathematical function description of the data packet impulse.

Definitions of cable (axon) impedance and structural return loss, in the most general terms, is the ratio of the voltage to current of a signal traveling in one direction down the cable. In coaxial cable (axon), the value of the impedance will depend upon the ratio of the inner and outer conductor diameters, and the dielectric constant of the material between the inner and outer conductors. The value of the conductivity will affect the impedance to the extent that RF signals (data packet impulses) do not travel on the surface of the conductor, but propagate into the conductor by what is known as the skin depth. The finite conductivity also causes losses that increase with RF frequency (data packet impulses' Fourier series), and this can change the effective cable impedance. Finally, the construction of the cable (axon) can change along the length of the cable (axon), with differences in conductor thickness, dielectric material, and outer conductor diameter changing due to limitations in manufacturing. Thus the cable (axon) impedance can vary along the length of the cable (axon). The extent to which the manufacturing imperfections degrade cable (axon) performance is characterized by the specification structural return loss (or SRL). Structural return loss is the ratio of incident signal to reflected signal in a cable (axon). This definition implies a known incident and reflected signal. In practice, the SRL is loosely defined as the reflection coefficient of a cable (axon) referenced to the cable (axon) impedance. The reflection seen at the input of a cable (axon), which contributes to SRL, is the sum of all the tiny reflections along the length of the cable (axon). In terms of cable (axon) impedance, the SRL can be defined mathematically as: ρSRL(ω)=eq. 1 Zin (ω)−Zcable (Axon) Zin (ω)+Zcable (axon). Zin is the impedance seen at the input of the cable (axon), and Zcable (axon) is the nominal cable (axon) impedance. Cable (axon) impedance is a specification that is defined only at a discrete point along the cable (axon), and at a discrete frequency. However, when commonly referred to, the impedance of the cable (axon) is some average of the impedance over the frequency of interest. Structural return loss, on the other hand, is the cumulative result of reflections along a cable (axon) as seen from the input of the cable (axon). The above definitions need to be expressed in a more rigorous form in order to apply a measurement methodology. One definition of cable (axon) impedance is that impedance which results in minimum measured values for SRL reflections over the frequency of interest (data packet impulse via Fourier series). This is equivalent to measuring a cable (axon) with a return loss bridge that can vary its reference impedance. The value of reference impedance that results in minimum reflection, where minimum must now be defined in some sense, is the cable (axon) impedance. Mathematically, this is equivalent to finding a cable (axon) impedance Zcable (axon) such that: eq. 2[ρ(ω, Zcable (axon))] ∂(Zcable (axon))=0 where ρ(ω) is some mean reflection coefficient. Thus, cable (axon) impedance and SRL are somewhat inter-related; the value of SRL depends upon the cable (axon) impedance, and the cable (axon) impedance is chosen to give a minimum SRL value. An alternate definition of cable (axon) impedance is the average impedance presented at the input of the cable (axon) over a desired span. This can be represented as Zavg=eq. 3 Fmin ∫Fmax Zin (ω)d ω2π(Fmax−Fmin). The value found for Zavg would be substituted for Zcable (axon) in equation (1) to obtain the structural return loss from the cable (axon) impedance measurement. Any discourse on cable (axon) measurements should include a discussion of the unique qualities of cables (axons) that make measurements so challenging. Because cables (axons) are electrically very long, and very low loss, the effect of any periodic defect in the cable (axon) will be greatly multiplied. Periodic faults and SRL-SRL is a reflection of incident energy that is caused by disturbances (bumps) in the cable (axon) which are distributed throughout the cable (axon) length. These bumps may take the form of a small dent, or a change in diameter of the cable (axon). These bumps are caused by periodic effects on the cable (axon) while in the manufacturing process (or myelination process). For example, consider a turn-around wheel with a rough spot on a bearing. The rough spot can cause a slight tug for each rotation of the wheel. As the cable (axon) is passed around the wheel, a small imperfection can be created periodically corresponding to the tug from the bad bearing. Each of these small variations within the cable (axon) causes a small amount of energy to reflect back to the source due to the non-uniformity of the cable (axon) diameter. Each bump reflects so little energy that it is too small to observe with fault location techniques. However, reflections from the individual bumps can sum up and reflect enough energy to be detected as SRL. As the bumps get larger and larger, or more of them are present, the SRL values will also increase. The energy reflected by these bumps can appear in the return loss measurement as a reflection spike at the frequency that corresponds to the spacing of the bumps. Discrete cable (axon) faults and SRL—reflections from faults within the cable (axon) will also increase the level of SRL measured. The energy reflected from a fault will sum with the energy reflected from the individual bumps and provide a higher reflection level at the measurement interface.

The brain operates as a mesh network utilizing the multiple ends of its singular axon to interface via synapses to multiple neurons. A mesh network is a network topology in which each node relays data for the network. All mesh nodes cooperate in the distribution of data in the network. Mesh networks can relay messages using either a flooding (broadcast) technique or a routing technique. With routing, the properly addressed message is propagated along a path by hopping from node to node (neuron to neuron) until it reaches its proper addressed destination. To ensure all its paths' availability, the network must allow for continuous connections and must reconfigure itself around broken paths, using self-healing algorithms such as Shortest Path Bridging. Self-healing allows a routing-based network to operate when a node breaks down or when a connection becomes unreliable. As a result, the network is typically quite reliable, as there is often more than one path between a source and a destination in the network. A mesh network whose nodes (neurons) are all connected to each other is a fully connected network. Fully connected wired networks have the advantages of security and reliability, i.e. problems in a single cable affect only the two nodes attached to it. However, in such networks, the number of cables, and therefore the cost, goes up rapidly as the number of nodes increases. Fortunately, the human brain's 100 billion plus neurons and associated axons and dendrites form the most complex “known to man” mesh network for the costs of two humans' love, desire to procreate and their patience to raise the freshly constructed brain mesh network owner(s). The patience part may be the most interesting! With the complexity of the brain's mesh network of neuron/axon/dendrite connections, it is obvious that either an occasional or a continuous and massive internal generation of ill addressed data packets from a strategically but, unfortunately positioned axon defect produces the overwhelming data packet flow consisting of the rogue “ill-addressed” message packets flowing from one neuron to the next to never find its destination plus the properly addressed message packets that maintain the only portion of reality that the patient of schizophrenia has to hold on to.

Prior art research of the neurological medical community has produced an understanding that schizophrenia is a developmental disorder that involves abnormal connectivity. The evidence is overwhelming. Doctors have always wondered why schizophrenia typically develops during adolescence. Recall that adolescence is the period when the forebrain is being myelinated. The neurons there have been established, however, the myelin of the neurons' axons is still in the formative stages. Prior art studies by the neurological medical community have concluded that axons are abnormal (possessing fewer oligodendrocytes than normal) in several regions of the schizophrenic brain. Also, the prior art studies discovered that many mutated or damaged genes linked to schizophrenia were involved with myelin formation. Axon abnormalities have also been found in people affected by ADHD, bipolar disorder, language disorders, autism, dyslexia, tone deafness and others. Cognitive function depends on neuronal communication across synapses in the cortex's gray matter, where most psychoactive drugs act to diminish the symptoms of schizophrenia. Optimal communication among brain regions, depends on the axonal matter connecting the regions without any disruptions or confusion resulting from extraneous data flow. It has been found in prior art studies that disruption of genes in oligodendrocytes causes striking behavioral changes that mimic schizophrenia. The behavioral effects involve one of the same genes, neuregulin, found to be abnormal in biopsies of schizophrenic brains. This novel process and method to prevent and or cure schizophrenia and other mental illnesses may be accomplished by detecting the abnormal or damaged gene(s), neuregulin and other associated genes, of the schizophrenia patient or even scan young people for the abnormal or damaged myelination gene(s), neuregulin and other associates genes, and utilize gene splicing and or other gene correcting methods to put into place and activate the newly installed proper gene(s) to produce the proper proteins for the memory dendrite paring and final and proper axon myelination.

Although the present invention is illustrated and described herein with reference to preferred embodiments and specific examples thereof, it will be readily apparent to those of ordinary skill in the art that other embodiments and/or examples may perform similar functions and/or achieve like results. All such equivalent embodiments and examples are within the spirit and scope of the present invention, are contemplated thereby, and are intended to be covered by the following non-limiting claims.

1. A method for treating schizophrenia and other forms of mental illness, comprising:
given a brain comprising neurons coupled by an axon comprising an inner core and an outer myelin sheath, and given one or more defects in the outer myelin sheath, repairing the one or more defects in the outer myelin sheath with one or more of a protein and a lipid such that the outer myelin sheath has a substantially constant electrical impedance for the transmission of data energy between the neurons.

2. The method of claim 1, wherein the one or more defects in the outer myelin sheath are repaired with one or more of the protein and the lipid such that data energy is not undesirably reflected from the direction of a receiving neuron in the direction of a transmitting neuron within the axon.

3. The method of claim 1, wherein the one or more defects in the outer myelin sheath are repaired with one or more of the protein and the lipid such that data energy is not undesirably reflected from the direction of a receiving neuron in the direction of a transmitting neuron within the axon, thereby causing the transmitting neuron to confuse the reflected data energy with expected reply data energy from the receiving neuron responsive to transmit data energy.

4. The method of claim 1, further comprising first determining that one or more of a neuregulin level and a gene are abnormal in the brain.

5. The method of claim 4, further comprising repairing the one or more defects in the outer myelin sheath with one or more of the protein and the lipid by controlling the neuregulin level during myelination of the axon.

6. The method of claim 4, further comprising repairing the one or more defects in the outer myelin sheath with one or more of the protein and the lipid via one of gene splicing and gene repair during myelination of the axon.

7. A system for treating schizophrenia and other forms of mental illness, comprising:
given a brain comprising neurons coupled by an axon comprising an inner core and an outer myelin sheath, and given one or more defects in the outer myelin sheath, means for repairing the one or more defects in the outer myelin sheath with one or more of a protein and a lipid such that the outer myelin sheath has a substantially constant electrical impedance for the transmission of data energy between the neurons.

8. The system of claim 7, wherein the one or more defects in the outer myelin sheath are repaired with one or more of the protein and the lipid such that data energy is not undesirably reflected from the direction of a receiving neuron in the direction of a transmitting neuron within the axon.

9. The system of claim 7, wherein the one or more defects in the outer myelin sheath are repaired with one or more of the protein and the lipid such that data energy is not undesirably reflected from the direction of a receiving neuron in the direction of a transmitting neuron within the axon, thereby causing the transmitting neuron to confuse the reflected data energy with expected reply data energy from the receiving neuron responsive to transmit data energy.

10. The system of claim 7, further comprising means for first determining that one or more of a neuregulin level and a gene are abnormal in the brain.

11. The system of claim 10, further comprising means for repairing the one or more defects in the outer myelin sheath with one or more of the protein and the lipid by controlling the neuregulin level during myelination of the axon.

12. The system of claim 10, further comprising means for repairing the one or more defects in the outer myelin sheath with one or more of the protein and the lipid via one of gene splicing and gene repair during myelination of the axon.
